(19) United States
(12) Patent Application Publication
(10) Pub. No.: US 2013/0109964 A1
(43) Pub. Date: May 2, 2013

(54) METHODS AND APPARATUS FOR ANALYZING MEDICAL IMAGING DATA

(71) Applicant: Matthew David Kelly, Botley (GB)

(72) Inventor: Matthew David Kelly, Botley (GB)

(21) Appl. No.: 13/651,755

(22) Filed: Oct. 15, 2012

(30) Foreign Application Priority Data
Oct. 14, 2011 (GB) 1117810.0

(51) Int. Cl. A61M 5/00; G06K 9/46

(52) U.S. Cl. CPC A61M 5/007; G06K 9/46
USPC 600/431; 382/131

(57) ABSTRACT

In a method and apparatus for analyzing medical imaging data of a subject from an imaging modality using a tracer in which a characteristic of the tracer varies with time, are disclosed, a region of interest in a scanned image volume is determined. Data are then obtained from detection of tracer emission events in the scanned imaging volume, and from the data those events which originated in the region of interest are determined. A time series of emission events for the region of interest is then recorded.

### BACKGROUND OF THE INVENTION

1. Field of the Invention

This invention is directed to methods and apparatus for analyzing medical imaging data of a subject from an imaging modality using a tracer in which a characteristic of the tracer varies with time.

2. Description of the Prior Art

In the medical imaging field, several nuclear medicine emission imaging schemes are known. For example PET (Positron Emission Tomography) is a method for imaging a subject in 3D using an ingested radio-active substance which is processed in the body, typically resulting in an image indicating one or more biological functions. FDG, for instance, is a glucose analog which is used as the radiopharmaceutical tracer in PET imaging to show a map of glucose metabolism. For cancer, for example, FDG is particularly indicated as most tumors are hypermetabolic, which will appear as a high intensity signal in the PET image. For this reason, PET imaging is widely used to detect and stage a wide variety of cancers. The level of glucose activity is usually highly correlated with the aggressiveness and extent of the cancer, and, for example, a reduction in FDG signal between a baseline and a follow-up scan is often indicative of a positive response to therapy.

A key criterion used in evaluating suspicious lesions in a PET scan is the Standardized Uptake Value (SUV). This value is computed from the number of counts of emission events recorded per voxel in the image reconstructed from the event data captured in the PET scan (coincidence emission events along the line of response). The SUV value can also, for example, be adjusted with the intention of accounting for differences in body mass/composition and concentration of radiotracer injected. Effectively, the SUV's purpose is to provide a standardized measure of the spatial distribution of radiotracer concentration throughout the imaged portion of the body.

The concentration of radiotracer accumulating in any given tissue region in the body is dependent upon both the affinity of that tissue region for the tracer and the supply of tracer to that tissue region.

Conventionally, PET scans are acquired using a static protocol, producing a single image volume representing the average counts detected over a fixed period of time following a given interval between radiotracer injection and image acquisition.

The interval between radiotracer injection and PET acquisition is intended to allow the system to reach a steady state equilibrium, with respect to radiotracer distribution. However, with most clinical protocols using an interval of 45-60 mins for 18F-FDG, this equilibrium is often not achieved, resulting in under estimation of metabolic rate for the malignancies. In addition, static imaging prior to equilibrium can, in some cases, make differentiation between tissues having distinct tracer uptake profiles (e.g. malignant and inflamed) difficult (FIG. 1).

FIG. 1 is a schematic illustration of consequence of imaging before equilibrium. It is known that over the first two hours after the injection of FDG, malignant cells will continue to take up FDG whereas inflamed cells will take up FDG and then wash it out progressively (or at least plateau). In FIG. 1, these time-activity curves represent schematically the different uptake patterns over time of FDG in cancer cells (108) and inflamed cells (110). The two dashed vertical lines (102, 104) represent the beginning and end of the scanning time and the textured pattern in between (106) represents the time during which data is acquired to generate an image. In this situation, intensity alone (i.e., mean activity measured during acquisition) would not allow differentiation of cancer from inflammation.

Three protocols that could be used to differentiate tissues (e.g., tumor vs. inflammation) based on rate of change of tracer uptake are:

1) Dynamic protocol: Scan is acquired from time of tracer injection with acquired data temporally binned to allow measurement of time activity curves (TACs). Pharmacokinetic analysis or clustering techniques can then be applied to differentiate the different tissue types; however, these scans can take a long time (up to 2 hours) and are therefore not typically performed in a clinical setting.

2) Dual time point scan: Two scans are obtained at different time points after injection, e.g., after 60 and 90 minutes. The change in measured uptake between the two scans can then be used to differentiate tissue types with different uptake profiles; however, these protocols are also often time consuming and not routinely used in a clinical environment.

3) Slope from static: A derivative image is either reconstructed directly or computed from a rebinned static acquisition protocol, such as described in UK patent application no. GB2464212. The computed rate of change of tracer uptake can then be used to differentiate tissue types. This approach avoids the additional time burden associated with the previous two approaches; however, using reconstructed data to compute slope from a rebinned static acquisition can introduce significant error into the computation since each of the rebinned volumes are reconstructed independently and subject to considerable noise due to the short frame durations.

Other problems arise in pharmaco-kinetic modeling, the method whereby the image is acquired dynamically over a period of time in order to obtain a series of images which reflect the uptake pattern of the tracer over the whole body at many instants after the injection of the tracer. The modeling is based on the hypothesis of basic diffusion of the tracer between various tissues (modeled as “compartment”). The parameters defining the diffusion rates can be estimated from the image data. The equations defining these diffusion processes can be solved only with the knowledge of the “input function” of the system: in that case, it is necessary to know the amount of concentration over time of tracer in the blood (which brings the tracer to the tissue).

This “blood input function” (BIF) is difficult to compute as the tracer is usually injected very quickly: less than 30 second injections, but often, as a fast bolus. As a consequence, the concentration of tracer in the blood at a particular location starts from zero, then increases very sharply for a short period of time and then fades out as the tracer diffuses in the entire blood stream and is taken up by the tissue.

FIG. 3 illustrates the concentration of tracer over time (302) at a particular location: first, a sharp increase (304) as the bolus passes through, then a slower decrease (306) as the tracer diffuses in the blood stream and is taken up by the tissue.

The BIF can be obtained using several ways:

1) arterial sampling: some blood samples are taken from the patient and the activity in each drop of blood is counted in a “well counter” (radioactivity measurement device). This is accurate, but fairly impractical for clinical use (blood being drawn from patients) or pre-clinical (small animals do not have enough blood);

2) image derived input function: the BIF is calculated from the image.

Various methods can be used:

a. calculation from a region of interest (ROI). The ROI is placed in an area where an artery is located (carotid, aorta, or left ventricular blood pool). This can be done, but the estimation suffers from partial volume effect due to the generally small volume of the artery (especially if the organ of interest is not close to the heart);

b. statistical modeling of the BIF using Independent Component Analysis (ICA) or Factor Analysis (FA). Such methods try to describe the set of Time Activity Curves (TACs) as a linear combination of “independent” TACs, one of which is believed to be the BIF. Although the methods are promising, there is no valid justification for one of these independent TACs to be the BIF. Moreover, the number of independent component TACs need to be defined in advance to the processing, and the resulting estimated BIF depends on that number;

3) sinogram based techniques: in order to reduce the partial volume effect, some techniques using direct Region of Interest reconstruction [2,3]: the methods calculate the mean value within a pre-defined ROI directly based on the sinogram data. This has the advantage of being more accurate and less biased, but still relies on the creation of sinogram data and binning the time information from frames;

4) Nichols et al. “Spatial reconstruction of list-mode PET data”, IEEE Trans Med Im 2002 discloses a method for reconstructing the TACs at a particular point directly. The method reconstructs a dynamic representation of the image as a continuous data. The advantage is that the information is smooth. Should the location of a suitable ROI where the BIF can be measured, the BIF would be a continuous function of time. However, the method still falls in the drawback of classic reconstruction methods (partial volume, spill over).

### SUMMARY OF THE INVENTION

An object of the present invention is to address the above-described problems and to provide improvements upon the known devices and methods.

The above object is achieved in accordance with the present invention by a method and apparatus. The method and apparatus and storage medium according to the invention allow the use of the raw data from the medical imaging modality to be used to estimate various factors, rather than data reconstructed from that raw data.

The present invention also encompasses a non-transitory, computer-readable data storage medium encoded with programming instructions that, when the storage medium is loaded into a computerized control and evaluation system of an imaging modality, cause the system to execute one or more embodiments of the method according to the invention.

The above aspects and embodiments may be combined to provide further aspects and embodiments of the invention.

### BRIEF DESCRIPTION OF THE DRAWINGS

FIG. 1 illustrates change in uptake for different tissues.

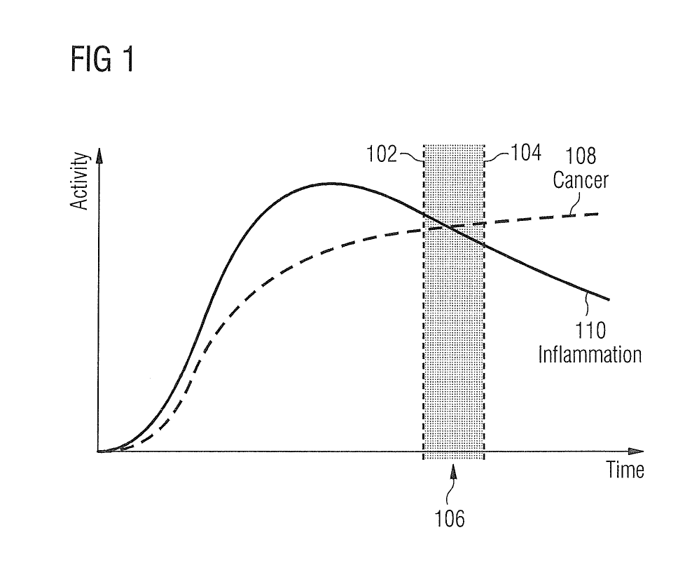

FIG. 2 illustrates a method of image processing according to an embodiment of the invention.

FIG. 3 illustrates tracer concentration over time in a subject.

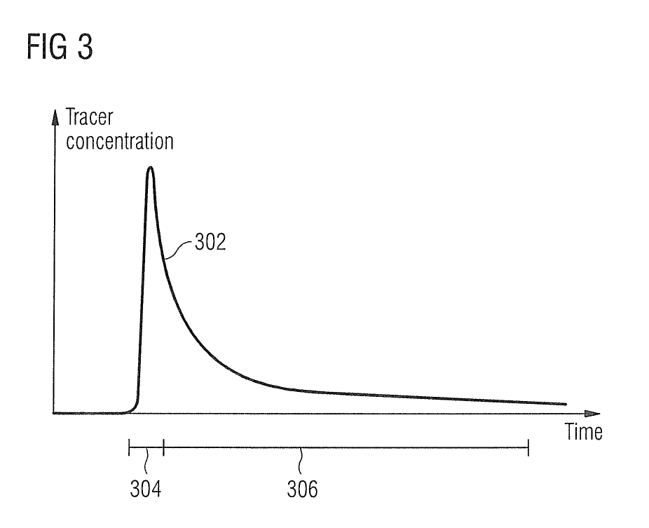

FIG. 4 illustrates a method of image processing according to an embodiment of the invention.

FIG. 5 illustrates a result of image processing according to an embodiment of the invention.

FIG. 6 illustrates tracer concentration according to an embodiment of the invention.

FIG. 7 is a block diagram illustrating the basic components of an apparatus according to an embodiment of the invention.

### DESCRIPTION OF THE PREFERRED EMBODIMENTS

When the following terms are used herein, the accompanying definitions can be applied:

PET—Positron Emission Tomography

ROI—Region of Interest

VOI—Volume (Region) of Interest

FDG—2-18F-Fluoro-2-deoxy-D-glucose

AUC—Area Under the Curve

SUV—Standardized Uptake Value

TAC—Time-Activity Curve

CT—Computed Tomography

LOR—Line of Response

BIF—Blood Input Function

LM—List Mode (raw PET data recording each individual photon detection)

Embodiments of the invention seek to use the raw data from the imaging modality, rather than reconstructed data, in order to find a time series of events which can be used to estimate further factors. The time series may be to inaccurate for use in reconstruction, but is sufficiently accurate for estimation of useful factors, without the distorting effects of reconstruction on those estimations,

For example, in deriving a rate of change of uptake, reconstruction noise can be avoided by reconstructing a derivative image by the method described in the following sections.

Another embodiment defines a method which obtains the BIF directly from the list mode data without having to reconstruct the data, escaping the partial volume, spill over and time binning which is needed when reconstructing an image.

One embodiment of the inventive method uses the reconstructed PET volume to identify the region of interest (ROI) and compute an average uptake for that ROI. The rate of change of tracer uptake for that ROI however, is computed directly from the Listmode (LM) data.

LM data is a file containing all detections of photons coming from the positron disintegrations. The LM data contains the true events, but also some random events (events which do not correspond to a single positron disintegration, but to photons originating from separate positron disintegrations detected at the same time) and scatter events (pairs of photons for which at least one photon has been scattered by the body tissues to create an erroneous line of response).

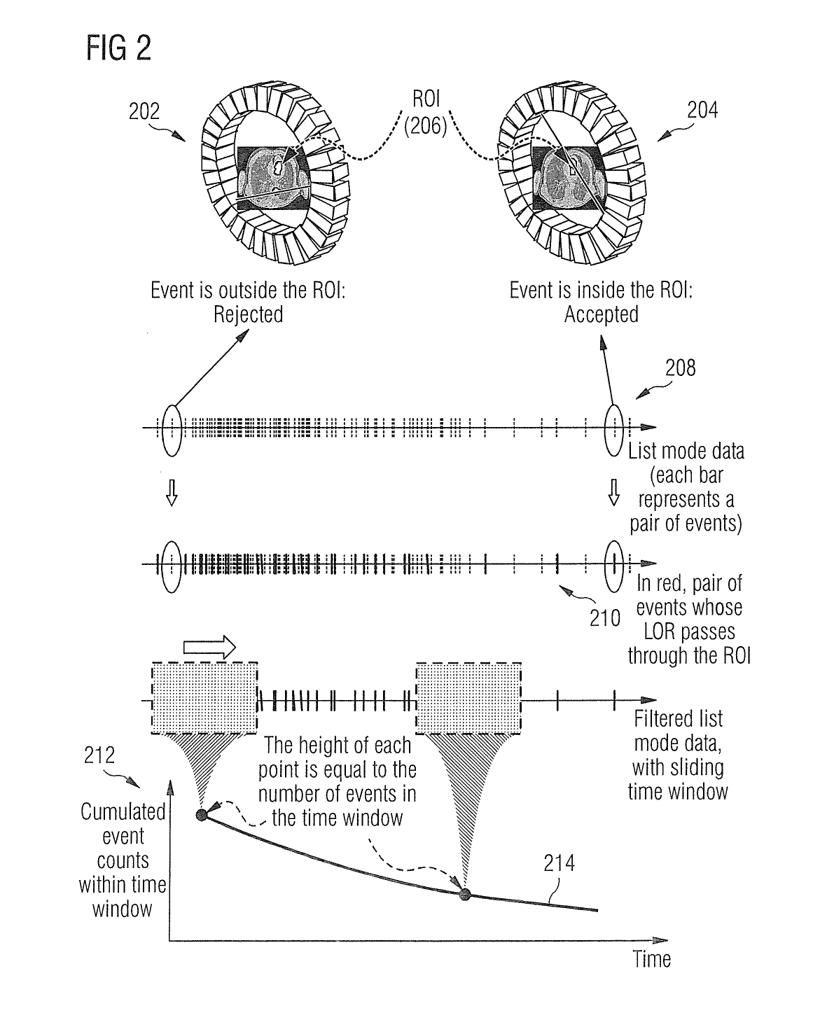

From the list-mode, the pairs of detected events whose lines of response (LOR) pass through the ROI in the reconstructed image are identified (as explained elsewhere herein). The change in frequency of such events over time can then be computed using, for example, a sliding time window (FIG. 2). The rate of change of uptake can then be estimated directly from this plot of frequency against time, for example using linear regression.

Since only the rate of change of uptake is estimated from the LM data (the average uptake is measured from the reconstructed image), the effects of random and scatter events should be small (as compared to their effect on the absolute value) since these contributions should generally be relatively constant over the duration of the scan for a given body region.

In situations where the contribution from randoms and scatter to the estimated rate of change is substantial, corrections for randoms and scatter may be performed on the LM data. This could be achieved with the same techniques used for correcting sinograms prior to reconstruction, with the corrections to the sinogram bins propagated back to the corresponding LM events.

Referring to FIG. 2, this illustrates detection of an event 202 which is outside the ROI (206), and of an event 204 inside the ROI 206. This list mode data (208) from the scan is obtained, and all events (210) whose line of response passed through the ROI are accepted, others rejected.

The time series is then recorded (212). In this example, a sliding time window is used, on only those events in the ROI. The number of events in the window is counted as the window moves, giving a plot of the counts over time. This time series/plot (214) can then be used to calculate the rate of change of uptake in the ROI.

In alternatives, as opposed to using a sliding window to generate the plot of event frequency against time, the data could simply be divided into a series of contiguous bins from which the slope could be calculated.

In another alternative, for lesions close to sites of high physiological uptake, i.e., bladder or heart, which may dominate the signal from the ROI, those events whose LOR also pass through the region of high physiological uptake could be excluded. This would remove the contribution from the site of high physiological uptake. The regions of high physiological uptake could be identified from the reconstructed PET image.

Features of this embodiment of the invention may include:
estimating the rate of change of tracer uptake in a PET scan directly from the acquired list mode data by the following steps:
   define the region of interest in the reconstructed image
   identify all events whose line of response passes through said region of interest;
   exclude lines of response passing through other regions of high uptake;
   if necessary, perform corrections for scatter and random events;
   measure rate of change of frequency of said events over duration of scan

In the other embodiment outlined above, for obtaining the BIF, again the proposed embodiment only needs the information of where the ROI is, for instance, from the structural image that is acquired at the same time of the PET-CT (in that case, the image would be a CT, but for MR-PET devices, the image would be MRI). The ROI could be defined either as a set of CT or MR voxels, or as a 3D shape defined with a mesh.

Again, from the list-mode, first the pairs of detected events are kept (again called “events”). Each event is filtered if the “event” corresponds to a line of response which goes through the BIF ROI, it is kept, otherwise, it is rejected.

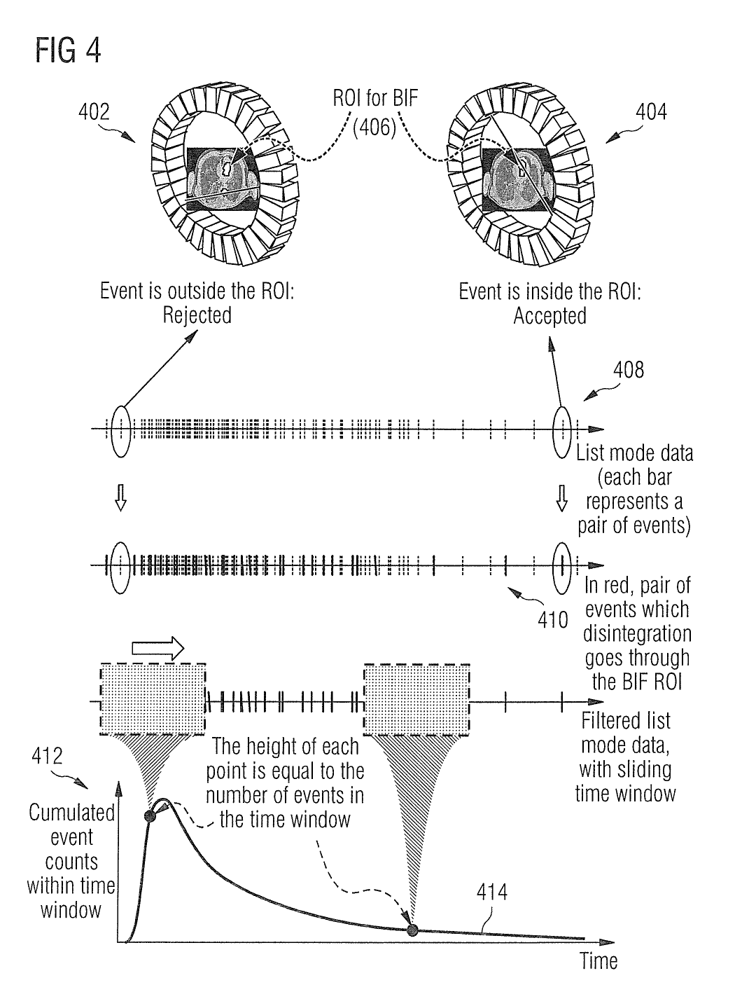

Referring to FIG. 4, a similar process to that in the previous embodiment is undertaken, for detection of an event 402 which is outside the ROI (406), and of an event 404 inside the ROI 406. In this case, the ROI is of course typically chosen as a significant area of the blood pool, such as an artery.

The list mode data (408) from the scan is obtained, and all events (410) whose line of response passed through the ROI are again accepted. The time series is then recorded (412). Again in this example, a sliding time window is used, on only those events in the ROI. Here, the number of events in the window is counted as the window moves, giving a plot of the counts over time. This time series/plot (414) can then be used as an estimate of the BIF, because it gives an accurate measure of the frequency of events in the blood pool ROI.

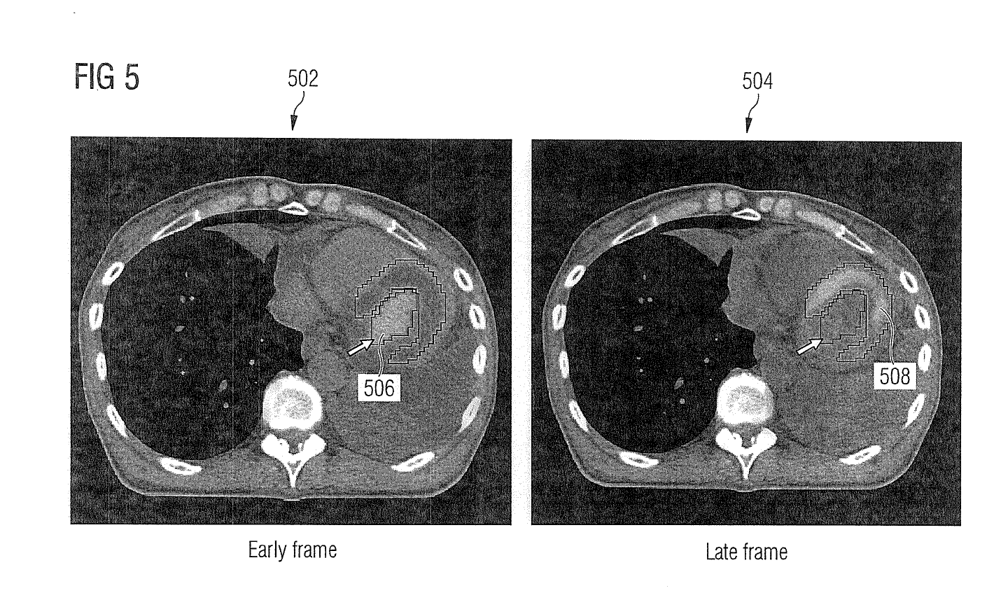

FIG. 5 shows regions of interest (506, 508) in early (502) and late (504) frames of the PET data, used to derive TACs from the list mode data. The blood pool region is placed in the LV cavity (506). The example tissue ROI (508) is in the myocardium (in that example, that tissue ROI surrounds a great part of the LV cavity).

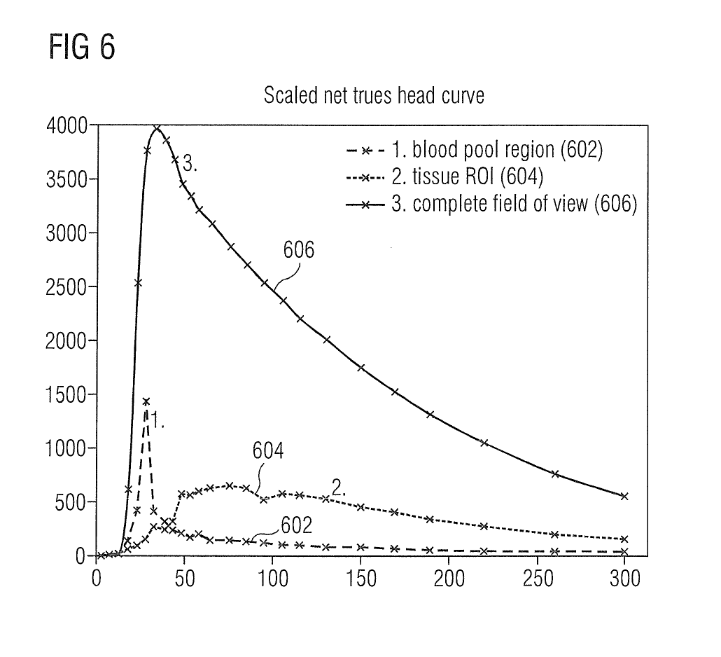

FIG. 6 shows an example of TACs derived from a blood pool region (602) and a tissue region (604) from the list-mode data, along with the head curve (606) which is the curve counting all events in the field of view.

When the tracer has just been injected, most activity is coming from the blood pool, rather than from other parts of the body along the line of response: therefore, the early part of the blood pool TAC is not much polluted by extra-ROI activity, and less biased from the true activity from the ROI.

This embodiment has a number of advantages and possible drawbacks:
advantages:
it is fast to compute: the condition of the intersection with the ROI is simple to implement.
It does not rely on the reconstruction of an image, nor from the estimation of sinograms
possible drawbacks:
The condition of acceptance or rejection of an event is quite loose: there is no easy way to define whether the event comes from inside or outside the ROI if only the condition of intersection of the line of response is checked. However, this can be mitigated by using Time of Flight information to further examine where the event originated.
There is no filtering of scattered events: these however can be filtered by using energy measurements from the detectors, in order to improve the accuracy of the measure.

The BIF is expected to have a very high activity at the beginning of the scan, just after the bolus has passed. After that, the activity comes down fairly quickly. That peak is key for PK modeling, and is easily missed with the classic image based methods. At the beginning of the scan, there is not much tracer anywhere else in the body but in the arteries, so the line of responses which contribute to the counting mechanism are most probably coming effectively from the ROI itself.

Later in the scan, the tracer is taken up in the rest of the body and could contribute to the counting mechanism: however, the significance of the BIF at late stages of the scan is much less than at the beginning, so the effect on the PK modeling is minimal. If anything, this could be mitigated by weighting the BIF in the PK modeling using high weights at the beginning of the scan and lower weights as time goes by.

Working directly from the list mode data means that no correction has been made to the signal, such as attenuation correction, scatter correction or decay correction. All these corrections can of course be made a posteriori (if one assume that the ROI is small, which is the case for this application).

Features of this embodiment may include:
a method to derive blood input function information directly from the list mode data without the need for reconstruction. Such BIF can be processed thereafter for PK modeling or other processing.

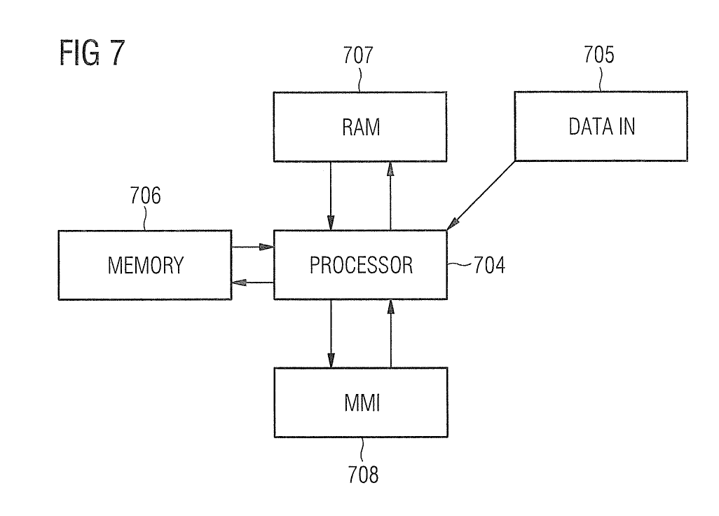

Referring to FIG. 7, the above embodiments of the invention may be conveniently realized as a computer system suitably programmed with instructions for carrying out the steps of the methods according to the invention.

For example, a central processing unit 704 is able to receive data representative of medical scans via a port 705 which could be a reader for portable data storage media (e.g. CD-ROM); a direct link with apparatus such as a medical scanner (not shown) or a connection to a network. The processor is configured to carry out steps such as determining a region of interest in a scanned image volume; obtaining data from detection of tracer emission events in the scanned imaging volume; determining from the data those events which originated in the region of interest; and recording a time series of emission events for the region of interest.

Software applications loaded on memory 706 are executed to process the image data in random access memory 707.

A Man-Machine interface 708 typically includes a keyboard/mouse/screen combination (which allows user input such as initiation of applications) and a screen on which the results of executing the applications are displayed.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method of analyzing medical imaging data of a subject acquired from an imaging modality using a tracer administered to the subject, in whom a characteristic of the tracer varies with time, comprising:
determining a region of interest in a scanned image volume;
obtaining data from detection of tracer emission events in the scanned imaging volume;
determining from the data those events which originated in the region of interest; and
recording a time series of emission events for the region of interest.

2. A method according to claim 1, wherein the step of determining the events which originated in the region of interest comprises determining those events for which the line of response passes through the region of interest.

3. A method according to claim 1, further comprising calculating from the time series a rate of change of emission events per unit time for the region of interest.

4. A method according to claim 3, further comprising comparing the rate of change of emission events per unit time for the region of interest with an expected behavior for a particular type of tissue of a scan subject.

5. A method according to claim 1, further comprising using the time series as an estimate of a blood input function for the scanned image volume.

6. A method according to claim 1, wherein the characteristic of the tracer is uptake of the tracer by tissue of the subject.

7. A method according to claim 6, further comprising calculating from the time series a rate of change of emission events per unit time for the region of interest, and calculating a rate of change of uptake of the tracer from the rate of change of emission events.

8. A method according to claim 7, comprising calculating the rate of change of uptake using linear regression.

9. A method according to claim 1, comprising employing PET as said imaging modality, and employing a radiopharmaceutical tracer as said tracer.

10. A method according to claim 1, comprising employing, as said data from detection of tracer emission events, list mode data from the scan, containing all detected pairs of emission events.

11. Apparatus for analyzing medical imaging data of a subject from an imaging modality using a tracer in which a characteristic of the tracer varies with time, comprising:
a processor configured to determine a region of interest in a scanned image volume; obtain data from detection of tracer emission events in the scanned imaging volume; determine from the data those events which originated in the region of interest; and record a time series of emission events for the region of interest; and
a display device configured to display a value from the time series with the region of interest.

12. A non-transitory, computer-readable data storage medium encoded with programming instructions, said storage medium being loaded into a computerized control and evaluation system of an imaging modality, said computerized control and evaluation system being provided with imaging data of a subject acquired from the imaging modality using a tracer administered to the subject, in which a characteristic of the tracer varies with time, said programming instructions causing said computerized control and evaluation system to:
determine a region of interest in a scanned image volume;
obtain data from detection of tracer emission events in the scanned imaging volume;
determine from the data those events which originated in the region of interest; and
record a time series of emission events for the region of interest.
